(19) United States
(12) Patent Application Publication
(10) Pub. No.: US 2006/0155585 A1
(43) Pub. Date: Jul. 13, 2006

(54) Image information processing apparatus, image information processing method and medical image information processing system

(75) Inventor: Tetsuya Onishi, Tokyo (JP)

Correspondence Address: FRISHAUF, HOLTZ, GOODMAN & CHICK, PC, 220 Fifth Avenue, 16TH Floor, NEW YORK, NY, 10001-7708, US

(73) Assignee: Konica Minolta Medical & Graphic, Inc., Tokyo (JP)

(21) Appl. No.: 11/326,812

(22) Filed: Jan. 6, 2006

(30) Foreign Application Priority Data
Jan. 13, 2005 (JP) JP2005-006213

(51) Int. Cl. G06F 19/00; A61B 5/00

(52) U.S. Cl. 705/3

(57) ABSTRACT

An object is to provide an image information processing apparatus, an image information processing method and a medical image information processing system wherein, when medical image information is shared by a plurality of hospitals, even patient ID and check ID given freely by each hospital can be registered correctly, and neither a patient mix-up nor impossibility of image registration is caused, and this image information processing method is one to preserve and administer medical images and to input medical images in other apparatuses to register them wherein, when inputting medical images in other apparatuses, image supplemental information determined in advance for preserved medical images is compared with that determined in advance for medical images in other apparatuses, and supplemental information is changed automatically so that the medical images in other apparatuses may be registered and administered properly based on the results of the comparison.

### FIELD OF THE INVENTION

The present invention relates to an image information processing apparatus wherein medical images in other apparatuses are inputted, preserved and registered, an image information processing method and a medical image information processing system.

### BACKGROUND OF THE INVENTION

It has been common in the past that supplemental information for a medical image such as, for example, patient ID or check ID is administered and registered in each hospital separately. On the other hand, as in TOKUKAI No. 2002-183319, there is proposed an information sharing method of a disease-diagnosis cooperative administration system wherein medical information is registered in a file server that shares medical information of patients, a limited patient information only is outputted when outputting medical information of the patient, and a medical institution is registered additionally in the disease-diagnosis cooperative administration system, for realizing information sharing for the disease-diagnosis cooperative administration system in a plurality of medical institutions which are connected by LAN and telephone lines.

However, even when trying to integrate pieces of information of medical images between hospitals to share them, data inconsistency is caused if no action is taken because patient ID and check ID are administered separately; namely, a different person is registered in the same patient and same check, or, on the contrary, the same person is registered in a different position, and further, even the registration itself is impossible. A measure to give a prefixal character which is peculiar to the patient ID for preventing the aforesaid problem is now tried in each hospital.

However, even if the prefixal character which is peculiar to the patient ID is given in each hospital, it becomes difficult to display or compare the previous images, and image retrieval also becomes difficult in the case of a different patient ID, because another patient is recognized for the display. Further, in the case of an information sharing server in Patent Document 1, its realization is actually difficult, except for the occasion where the registration is started from the beginning, or the occasion where each hospital that is sharing information has the same equipment or the equipment manufactured by the same manufacturer, and in addition, the work for reregistration requires considerable time and cost to be impracticable and to be difficult, because a huge number of medical images in the past are stored in each hospital and they need to be registered.

Further, when conducting image registration from an image preservation and administration apparatus (image server) to another image preservation and administration apparatus, necessary modifications cannot be made except the maker side, resulting in a time-consuming work. Even when a modification software is available, a user does not know how to modify to create data which can be registered and administered correctly.

### SUMMARY OF THE INVENTION

In view of the problems in the prior art stated above, an object of the invention is to provide an image information processing apparatus which can register patient ID and check ID each being given freely by each hospital when sharing medical image information between plural hospitals, and is free from a patient mix-up and from impossibility of image registration, an image information processing method and a medical image information processing system.

To attain the objected stated above, an image information processing apparatus of the invention is one that is equipped with a preservation means that preserves medical images and administers them and inputs medical images stored in other apparatuses and registers them in the preservation means, wherein there are provided a means to compare image supplemental information determined in advance for the preserved medical image preserved in the preservation means with that determined in advance for the medical image preserved in the other apparatus, when inputting the medical image stored in the other apparatus, and a means that changes automatically the supplemental information so that the medical images stored in other apparatuses may be registered and administered properly.

In the image information processing apparatus stated above, supplemental information for the preserved medical image is compared with that for the other apparatus, and when they are different each other, the supplemental information for the other apparatus can be changed automatically. Therefore, even in the case of the patient ID and check ID given freely by each hospital, they can be registered correctly, thereby, the image information processing apparatus is free from a patient mix-up and from impossibility of image registration. Further, even when the same patient is registered with different numbers in different hospitals, the patient can be handled in a unified way, and thereby, images and information in the past can simply be taken out. Further, it is possible to keep a huge amount of information stored in the past, without wasting them because the image information processing apparatus does not depend on equipment manufactured by the same manufacturers and on employment for numbering data. In addition, correction errors which can be caused by an operation of a user and a work time required for correcting a huge amount of stored data are reduced.

Another image information processing apparatus of the invention is one that is provided with a preservation means that stores and administers medical images and inputs medical images stored in other apparatuses and registers them in the preservation means wherein there are provided a means to compare image supplemental information of the preserved medical images preserved in the preservation means with that of medical images in the other apparatus, both being determined in advance, a means to display automatically candidate for change of the supplemental information that can register and administer the medical images in other apparatuses properly, and an operation means that conducts an indication of change for the supplemental information based on the results of the display.

In the image information processing apparatus stated above, supplemental information for the preserved medical image is compared with that for the other apparatus, and when they are different each other, a candidate for change of supplemental information of the medical image in other apparatus is displayed automatically, and an indication of change is given based on the result of the display, and the supplemental information can be changed. Therefore, even in the case of the patient ID and check ID given freely by each hospital, they can be registered correctly, thereby, the image information processing apparatus is free from a patient mix-up and from impossibility of image registration. Further, even when the same patient is registered with different numbers in different hospitals, the patient can be handled in a unified way, and thereby, images and information in the past can simply be taken out. Further, it is possible to keep a huge amount of information stored in the past, without wasting them because the image information processing apparatus does not depend on equipment manufactured by the same manufacturers and on employment for numbering data. In addition, correction errors which can be caused by an operation of a user and a work time required for correcting a huge amount of stored data are reduced. Further, when a user wishes to register a specific image or an image group only without changing it, for example, the user can do it because the user can give an indication of change based on the result of the display of a candidate for change of the supplemental information.

In each image information processing apparatus mentioned above, medical images in other apparatuses can be inputted through the on-line such as LAN or the Internet. It is also possible to input on an off-line basis by using a portable storage medium.

When returning the medical image and/or the supplemental information to other apparatuses in the case where at least any of an update, a change, an addition and elimination is carried out for the medical image and/or the supplemental information, it is preferable to provide a means to return only information changed for the registration. Owing to this, when returning medical images or supplemental information to other apparatuses, the medical images or the supplemental information can be registered under the situation before the change.

An image information processing method of the invention is one to preserve and administer medical images, and to input and register medical images stored in other apparatuses, wherein image supplemental information determined in advance for the preserved medical images is compared with that for the medical images in other apparatuses, and the supplemental information is changed automatically so that the medical information stored in other apparatuses may be registered and administered properly, based on the results of the comparison.

In the image information processing method stated above, supplemental information for the preserved medical image is compared with that for the other apparatus, and when they are different each other, the supplemental information for the other apparatus can be changed automatically. Therefore, even in the case of the patient ID and check ID given freely by each hospital, for example, they can be registered correctly, thereby, the image information processing method is free from a patient mix-up and from impossibility of image registration. Further, even when the same patient is registered with different numbers in different hospitals, the patient can be handled in a unified way, and thereby, images and information in the past can simply be taken out. Further, it is possible to keep a huge amount of information stored in the past, without wasting them because the image information processing apparatus does not depend on equipment manufactured by the same manufacturer and on employment for numbering data in the past. In addition, correction errors which can be caused by an operation of a user and a work time required for correcting a huge amount of stored data are reduced.

Another image information processing method of the invention is one that stores and administers medical images and inputs medical images stored in other apparatuses and registers them, wherein when inputting medical images in other apparatuses, image supplemental information determined in advance for the preserved medical images is compared with that for the medical images stored in other apparatuses, and a candidate for change of supplemental information that can register and administer the medical images in other apparatuses based on the result of the comparison, is displayed automatically, and an indication of change for the supplemental information is conducted based on the results of the display.

In the image information processing method stated above, supplemental information for the preserved medical image is compared with that for the other apparatus, and when they are different each other, a candidate for change of supplemental information for the medical images stored in other apparatuses is automatically displayed, and an indication of change is conducted based on the results of the display, and the supplemental information can be changed. Therefore, even in the case of the patient ID and check ID given freely by each hospital, for example, they can be registered correctly, thereby, the image information processing method is free from a patient mix-up and from impossibility of image registration. Further, even when the same patient is registered with different numbers in different hospitals, the patient can be handled in a unified way, and thereby, images and information in the past can simply be taken out. Further, itis possible to keep a huge amount of information stored in the past, without wasting them because the image information processing apparatus does not depend on equipment manufactured by the same manufacturer and on employment for numbering data in the past. In addition, correction errors which can be caused by an operation of a user and a work time required for correcting a huge amount of stored data are reduced. Further, when a user wishes to register a specific image or an image group only without changing it, for example, the user can do it because the user can give an indication of change based on the result of the display of the candidate for change of supplemental information.

In the image information processing method mentioned above, medical images in other apparatuses can be inputted through the on-line such as LAN or the Internet. It is also possible to input on an off-line basis by using a portable storage medium.

When at least any of an update, a change, an addition and elimination is carried out for the medical image and/or the supplemental information, it is preferable to output the medical images and/or the supplemental information to other apparatuses after returning back only information changed for the aforesaid registration. Owing to this, when returning the medical images and supplemental information to other apparatuses, they can be registered under the situation before the change.

A medical image information processing system of the invention is characterized to include the image information processing apparatus and the other apparatus. In the medical image information processing system, even in the case of the patient ID and check ID given freely by each hospital, for example, they can be registered correctly, thereby, the medical image information processing system is free from a patient mix-up and from impossibility of image registration. Further, even when the same patient is registered with different numbers in different hospitals, the patient can be handled in a unified way, and thereby, images and information in the past can simply be taken out. Further, it is possible to keep a huge amount of information stored in the past, without wasting them because the medical image information processing system does not depend on equipment manufactured by the same manufacturer and on employment for numbering data in the past. In addition, correction errors which can be caused by an operation of a user and a work time required for correcting a huge amount of stored data are reduced.

In the medical image information processing system mentioned above, the image information processing apparatus may be installed in a hospital that is different from another hospital where the other apparatus is installed. Incidentally, a plurality of other apparatuses may be installed respectively in a plurality of different hospitals.

In the image information processing apparatus, the image information processing method and the medical image information processing system of the invention, when medical image information is shared by medical institutions such as plural hospitals, even in the case of the patient ID and check ID given freely by each hospital, they can be registered correctly, resulting in no occurrence of problems of a patient mix-up and impossibility of image registration.

### BRIEF DESCRIPTION OF THE DRAWINGS

FIG. 1 is a block diagram showing schematically a medical image information processing system including an image information processing apparatus relating to the present embodiment.

FIG. 2 is a diagram showing schematically the structure of image information handled by the image information processing system shown in FIG. 1.

FIG. 3 is a diagram showing schematically the hierarchical structure of patient information and check information in DICOM standards.

FIG. 4 is a flow chart for illustrating operations of the image information processing apparatus shown in FIG. 1.

FIG. 5 is a flow chart for illustrating other operations of the image information processing apparatus shown in FIG. 1.

### DETAILED DESCRIPTION OF THE INVENTION

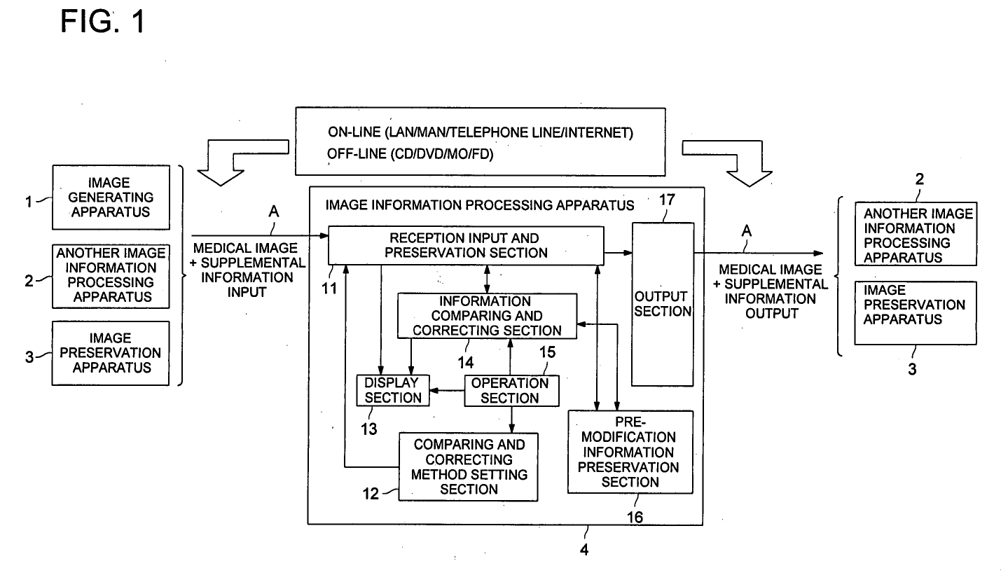

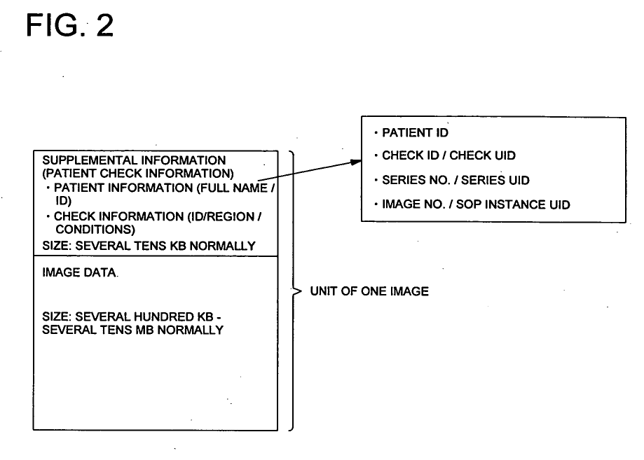

Preferred embodiments to practice the invention will be explained as follows, referring to the drawings. FIG. 1 is a block diagram showing schematically a medical image information processing system including an image information processing apparatus relating to the present embodiment. FIG. 2 is a diagram showing schematically the structure of image information handled by the image information processing system shown in FIG. 1.

The medical image information processing system shown in FIG. 1 is one that administers medical treatment information of a patient in each sectional system used by medical institutions, and it is especially one that can conduct a disease-diagnosis cooperative administration system among plural medical institutions which share medical treatment information of patients through LAN or telephone lines or through off-line.

The medical image information processing system shown in FIG. 1 is composed of image generating apparatus 1 that is composed of modality medical instruments such as CT, MRI and CR, and generates medical images through radiographing, image information processing apparatus 4 that can store, administer and register medial image information, image preservation apparatus 3 that stores, administers and registers medical image information and another image information processing apparatus 2 which is different from the image information processing apparatus 4.

In FIG. 1, image preservation apparatus 3 and another image information processing apparatus 2 are installed in a hospital that is different from a hospital where, for example, the image information processing apparatus 4 is installed. Meanwhile, the image generating apparatus 1 is installed in the same hospital where the image information processing apparatus 4 is installed.

In the medical image information processing system in FIG. 1, the image generating apparatus 1, another image information processing apparatus 2 and image preservation apparatus 3 are connected to the image information processing apparatus 4 through line A, so that medical image information may be inputted from each of apparatuses 1-3 to the image information processing apparatus 4, and medical image information may be outputted to apparatuses 2 and 3 from the image information processing apparatus 4.

Line A representing a connecting means in FIG. 1 is not restricted in particular, and it may be a telephone line or a private line, and also be connection by a network. The network may be LAN (Local Area Network) constructed, for example, in a single hospital, and LAN may be composed either of a single LAN segment or of plural LAN segments connected each other through routers (or gateways). Further, the network may be either WAN (Wide Area Network) composed of LANs of remote hospitals connected each other through private lines or a wide-area network such as the Internet. In FIG. 1, DICOM (Digital Imaging and Communications in Medicine) standard is used for communication of medical image information.

Further, an input of medical image information to the image information processing apparatus 4 may also be an off-line input by means of various types of portable storage media such as CD, DVD, MO and FD, in addition to an on-line input by means of the aforesaid line A.

The image information processing apparatus 4 can be composed of a personal computer, and it is equipped, as shown in FIG. 1, with reception input and preservation section 11 that receives, inputs and preserves medical image information transmitted from each of apparatuses 1-3, display section 13 that is composed of a liquid crystal panel and CRT for displaying inputted medical image information, operation section 15 that is composed of a pointing device such as a mouse with which a user selects necessary operations or inputs various types of information and of a key board, information comparing and correcting section 14 that retrieves registered supplemental information, then, compares with registered supplemental information for registering inputted medical image information and corrects and changes inputted supplemental information based on the result of the comparison and with output section 17 that outputs medical image information to respective apparatuses 2 and 3. The medical image information includes medical images and supplemental information concerning these images (see FIG. 2).

The image information processing apparatus 4 shown in FIG. 1 is further equipped with comparing and correcting method setting section 12 that establishes, in advance, a method for comparison and correction in the case of comparing, correcting and changing supplemental information in information comparing and correcting section 14 and with pre-modification information preservation section 16 that preserves supplemental information before the change for the supplemental information changed for registration in information comparing and correcting section 14.

Further, information comparing and correcting section 14 retrieves registered supplemental information, then, compares with registered supplemental information for registering inputted medical image information, and makes display section 13 to display candidate for correction of supplemental information, thus, a user can select appropriate candidate for correction from display of candidate for change of supplemental information, and to constitute to change to the selected information, by operating, for example, the confirmation button displayed on display section 13. Incidentally, it is also possible to construct an image plane of display section 13 so that a user can change candidate for correction freely by operation section on display section 13, for the occasion where the user judges the displayed candidate for correction not to be appropriate.

Next, image information inputted in the image information processing apparatus 4 and outputted from the image information processing apparatus 4 to image preservation apparatus 3 will be explained as follows, referring to FIG. 2. As shown in FIG. 2, medical image information is of a unit of a single image, and it is composed of image data and supplemental information concerning its image, and the image data is normally several hundred KB-several tens MB in size, while, supplemental information includes patient information such as a full name and patient ID and check information such as check ID, check regions and check conditions, and its size is normally about several tens KB. For each image, its supplemental information (patient check information) includes patient ID, check ID/check UID, series No./series UID and image No./SOP instance UID.

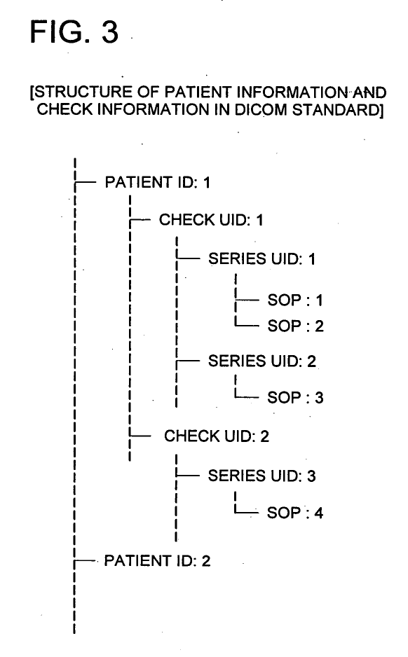

Next, structures of patient information in DICOM (Digital Imaging and Communications in Medicine) standard representing a communication protocol of medical image and of check information will be explained as follows, referring to FIG. 3. FIG. 3 is a diagram showing schematically the hierarchical structure of patient information and check information in DICOM standards.

As shown in FIG. 3, supplemental information includes patient information and check information, and it has the hierarchical structure in the order of patient ID→check UID→series UID→SOP. In this case, patient ID is ID for discriminating a patient exclusively, check UID is ID for discriminating a check exclusively, series UID is ID for discriminating series exclusively, and SOP instance UID is ID for discriminating an image exclusively.

In FIG. 3, if patient ID does not agree with a full name of a patient, an error is caused. An automatic input of a user of check UID is conducted by check ID. An automatic input of a user of series UID is conducted by series No. An automatic input of a user of SOP is conducted by image No.

Actual input is administered by each ID (patient ID/check ID/series No./image No.) in many cases. “ID, Number” include figures and characters, and the maximum number of their figures is ten-odd figures, and this is inputted by a user (example: 1234, A0001 and others). “UID” includes figures and periods and the maximum number of figures is as many as 64, and it is prepared mechanically with ID and numbers (example: 1. 2. 3456. 789 and others).

In information comparing and correcting section 14 in FIG. 1, retrieval and comparison are conducted for the supplemental information determined in advance, and the supplemental information includes the following items such as patient ID, check ID, series No., receipt number, check UID, series UID, image SOP instance UID, patient name, date of birth and sex distinction.

Information is taken out from the aforesaid information with an optional one or more combinations to conduct retrieval and comparison. A combination of information in the case of comparison can be established by comparing and correcting method setting section 12 in advance.

An inspection is generally made to find out whether wrong characters are used or not, whether a wrong character string is used or not, and whether a length of the character string is as specified or not (shorter/longer/fixed). However, it is not possible to check whether each discrimination number (ID and UID representing information for discrimination) agrees with the number which has already been registered in the present apparatus 4, in “patient-check-series-image”, if inputted data are not compared with data in the present apparatus 4.

Under the normal condition, the side on the apparatus 4 is administered by a hierarchical structure in FIG. 3 and a different one is allocated to each UID. Patient information and check information sent from other apparatus 2 in FIG. 1 are checked in apparatus 4 based on the structure mentioned above, and if the patient information and check information do not agree with the aforesaid structure, they are corrected in information comparing and correcting section 14 in FIG. 1. An example is as follows.

(A) For the same check ID as shown below, patient ID is different (the patient must be the same because the check is the same). (1) Patient ID: 2 check UID: 3 (which has been registered in apparatus 4) (2) Patient ID: 3 check UID: 3 (which has been inputted from other apparatus 2)

(B) For the same series UID as shown below, check UID is different (the check must be the same because the series is the same). (3) Check UID: 4 series UID: 12 (which has been registered in apparatus 4) (4) Check UID: 6 series UID: 12 (which has been inputted from other apparatus 2)

Next, a method of correction established in advance by comparing and correcting method setting section 12 for correcting and changing supplemental information in information comparing and correcting section 14 will be explained.

This method of correction is determined in advance in a way that no inconsistency is caused on registered information in apparatus 4, and correction is made as follows.

1. When operating under the fixed number of figures, the number of figures is increased or decreased: “Check ID: 0123”→“Check ID: 10123” or the like.

2. To insert to the forefront or the rearmost position a character that is never used: “Check ID: 0123”→“Check ID: A0123”.

3. To use a date or time to be revised: “Check ID: 0123”→“Check ID: 409011430”

4. To assign a number again newly (by using a character that is not doubled): “Check ID: 0123”→“Check ID: B0001” which is followed by “B0002”

In a viewpoint of the structure, when a higher structural portion is changed, the same change needs to be made for all images belonging to the subordinate structure in many cases. In such a case, actions can be taken as follows.

Before the change: A pattern where two images (SOP1, SOP2) exist in the same check and series (changing each UID or each ID) (1) Check UID: 1, series UID: 1, SOP: 1 (2) Check UID: 1, series UID: 1, SOP: 2

After the change: An image of (1) was selected, and check ID was changed from 1 to 2.

(3) Check UID: 2, series UID: 1, SOP: 1 (4) Check UID: 1, SOP: 2 ←

The check was changed to a different one undesirably despite the same series. Therefore, the change is made as follows.

After the change: In a right way, (5) Check UID: 2, series UID: 1 SOP: 1 (6) Check UID: 2, series UID: 1 SOP: 2 ← Automatic reflection was observed because the check was the same.

When check ID was corrected, if the original image is one in the same check, all numbers are automatically corrected to its check ID. In this way, when a higher structural portion is changed, the numbers in the subordinate structure are also changed automatically in the arrangement, so that no contradiction may be caused by the change in the higher structural portion.

By preserving the pre-modification patient ID and check ID as those mentioned above in pre-modification information preservation section 16 of the present apparatus 4, it is possible to return the necessary information to the state before the change, so that the patient ID and check ID may be registered when they are sent to apparatuses 2 and 3 in respective hospitals from the present apparatus 4. Incidentally, it is also possible to return to the state before the change, by adding the pre-modification information to a part of the changed image and image supplemental information.

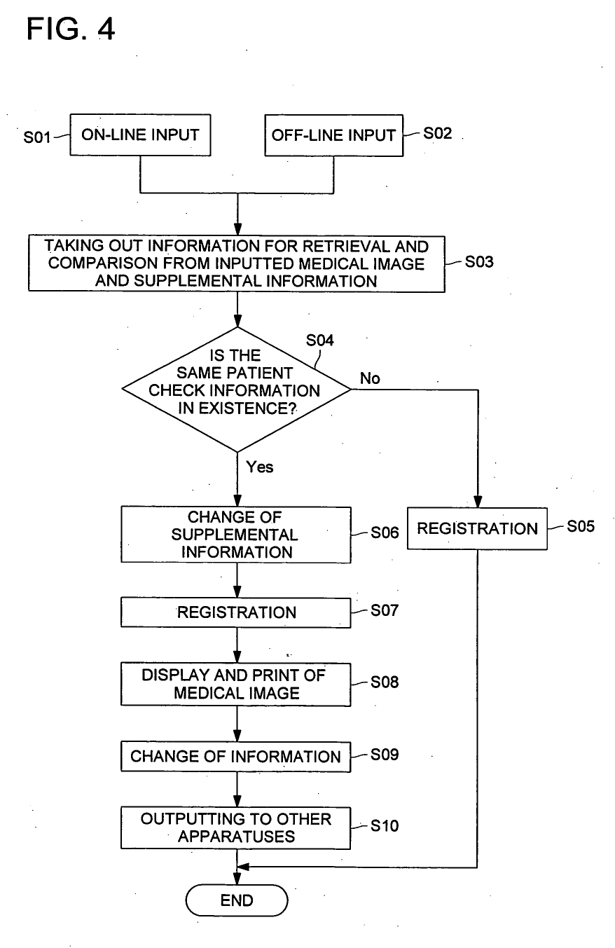

Operations of the image information processing apparatus 4 in the medical image information processing system shown in FIG. 1 will be explained next, referring to FIG. 4 which is a flow chart for illustrating operations of the image information processing apparatus in FIG. 1.

As shown in FIG. 1, medical image and its supplemental information are inputted first to reception input and preservation section 11 of the image information processing apparatus 4 from on-line media such as LAN, telephone lines, WAN and Internet or from off-line media such as CD, DVD, FD and MO. (SO1, SO2).

Next, in information comparing and correcting section 14 of the image information processing apparatus 4, information for retrieval and comparison determined in advance is taken out of the inputted medical image and supplemental information (SO3), and from that information, whether information of the same patient and same check is preserved and registered in the reception input and preservation section 11 of the image information processing apparatus 4 or not is searched, then, compared and confirmed (SO4), and when such information is not preserved, the information thus taken out is preserved in the reception input and preservation section 11 as it is, and is registered (SO5).

When the information of the same patient and same check is registered (SO4), the information is changed as stated above (SO6) so that no contradiction may be caused on patient information and check information, and is registered in the reception input and preservation section 11 of the image information processing apparatus 4 (SO7).

After the registration, medical image and information are displayed on display section 13, and the image is printed (SO8). In this way, the diagnosis can be made based on medical images.

Further, necessary information is changed (SO9) as occasion demands so that image and supplemental information corrected by a diagnosis or by a user may be registered in another apparatus 2 in the source of input, and the necessary information is outputted to another apparatus 2 (S10).

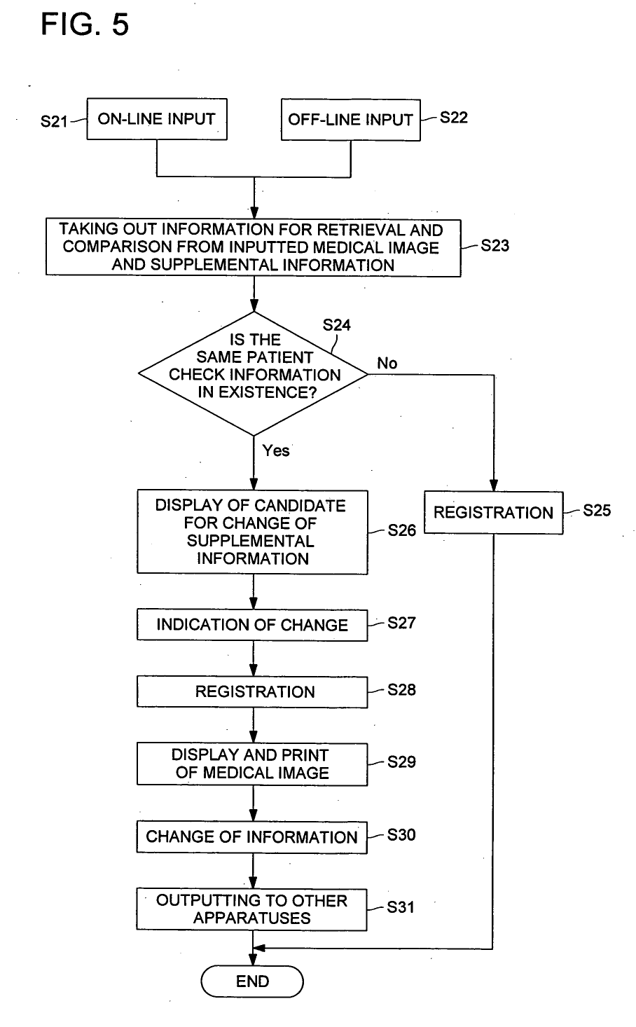

Next, other operations of the image information processing apparatus 4 in the medical image information processing system in FIG. 1 will be explained as follows, referring to FIG. 5 which is a flow chart for illustrating other operations of the image information processing apparatus in FIG. 1.

An operation in FIG. 5 is to display a candidate of correction of information which does not cause contradiction so that a user can select properly,

while in FIG. 4, information is changed automatically so that contradiction is not caused for patient information and check information in step SO6, when-information for check is registered.

As shown in FIG. 1, medical image and its supplemental information are inputted first to reception input and preservation section 11 of the image information processing apparatus 4 from on-line media such as LAN, telephone lines, WAN and Internet or from off-line media such as CD, DVD, FD and MO (S21, S22).

Next, in information comparing and correcting section 14 of the image information processing apparatus 4, information for retrieval and comparison determined in advance is taken out of the inputted medical image and supplemental information (S23), and from that information, whether information of the same patient and same check is preserved and registered in the reception input and preservation section 11 of the image information processing apparatus 4 or not is searched, then, compared and confirmed (S24), and when such information is not preserved, the information thus taken out is preserved in the reception input and preservation section 11 as it is, and is registered (S25).

When the information of the same patient and same check is registered (S24), a candidate of information which does not cause contradiction for patient information and check information is displayed on display section 13 (S26). A user changes supplemental information by confirming and selecting information to be changed from information displayed on display section 13, and by indicating the change of information on the display section 13 (S27), and registers the supplemental information in the reception input and preservation section 11 of the image information processing apparatus 4 (S28). Since the information for correction is displayed on display section 13, and information to be changed by a user can be selected on the display section 13 as stated above, this system can cope with an occasion where, for example, a user wishes to register a specific image or an image group only as it is without changing it.

After the registration, medical images and information are displayed on display section 13, and images are corrected, or prints are made from the images (S29). Thus, diagnoses can be made based on medical images.

Further, necessary information is changed (S30) as occasion demands so that image and supplemental information corrected by a diagnosis or by a user may be registered in another apparatus 2 in the source of input, and the necessary information is outputted to another apparatus 2 (S31).

As stated above, in the medical image information processing system shown in FIG. 1, when medical image information is shared by plural hospitals, even in the case of the patient ID and check ID assigned freely in each hospital, medical images and supplemental information from apparatus 2 of another hospital can be registered in image information processing apparatus 4 correctly, resulting in no occurrence of problems of a patient mix-up and impossibility of image registration. In addition, even when the same patient is registered in another hospital under the different number, the patient can be handled on a unified basis, thus, medical images and information in the past can be taken out in a simple and easy way, and information in a different hospital in the past can be utilized in a new diagnosis.

Further, in the image information processing apparatus 4, it is possible to keep a huge amount of information stored in the past, without wasting them because the image information processing apparatus does not depend on equipment manufactured by the same manufacturers and on employment for numbering data, and correction errors which can be caused by an operation of a user and a work time required for correcting a huge amount of stored data are reduced.

Though the preferred embodiments to practice the invention have been explained as stated above, the invention is not limited to them, and various modifications can be made within a scope of technical spirit of the invention. For example, in the aforesaid explanation of the present embodiment, though input and output of medical images and supplemental information are conducted between the image information processing apparatus 4 and another image information processing apparatus 2, it is naturally possible to conduct input and output of medical images and supplemental information between the image information processing apparatus 4 and image information processing apparatus 3 shown in FIG. 1, and it is also possible to conduct input from another image information processing apparatus 2 and to conduct output to the image information processing apparatus 3.

Further, in FIG. 1, it is naturally possible for the image information processing apparatus 4 to be connected to a different apparatus equal to apparatus 2 or 3 installed in still another hospital to conduct input and output.

1. An image information processing apparatus that has therein an image preservation means which stores and administers medical images, and inputs medical images in other apparatuses to register them in the image preservation means, wherein there are provided a means that compares image supplemental information determined in advance for the preserved medical images preserved in the image preservation means with that determined in advance for the medical images in the other apparatuses, when inputting medical images in other apparatuses, and a means that changes automatically the supplemental information so that the medical images in the other apparatuses may be registered and administered properly based on results of the comparison.

2. An image information processing apparatus that has therein an image preservation means which preserves and administers medical images, and inputs medical images in other apparatuses to register them in the image preservation means, wherein there are provided a means that compares image supplemental information determined in advance for the preserved medical images preserved in the image preservation means with that determined in advance for the medical images in the other apparatuses, when inputting medical images in other apparatuses, a means that displays automatically a change candidate of the supplemental information so that the medical images in the other apparatuses may be registered and administered properly based on results of the comparison, and an operation means that indicates changes of the supplemental information based on the results of the display.

3. The image information processing apparatus according to claim 1, wherein medical images in the other apparatuses are inputted on an on-line basis.

4. The image information processing apparatus according to claim 1, wherein medical images in the other apparatuses are inputted on an off-line basis.

5. The image information processing apparatus according to claim 1, wherein there is provided a means that returns only information changed for the registration to its original position when returning the medical images and/or the supplemental information to the other apparatus, in the case where at least any one of an update, a change, an addition and elimination is carried out, for the medical images and/or the supplemental information.

6. An image information processing method that preserves and administers medical images and inputs and registers medical images in other apparatuses, wherein image supplemental information determined in advance for the preserved medical images is compared with that determined in advance for the medical images in the other apparatuses, when medical images in other apparatuses are inputted, and the supplemental information is changed automatically so that the medical images in the other apparatuses may be registered and administered properly based on results of the comparison.

7. An image information processing method that preserves and administers medical images and inputs and registers medical images in other apparatuses, wherein image supplemental information determined in advance for the preserved medical images is compared with that determined in advance for the medical images in the other apparatuses, when medical images in other apparatuses are inputted, a candidate of changes for the supplemental information with which the medical images in other apparatuses can be registered and administered properly based on results of the comparison is displayed automatically, and an indication of the change for the supplemental information is carried out based on results of the display.

8. The image information processing method according to claim 6, wherein medical images in the other apparatuses are inputted on an on-line basis.

9. The image information processing method according to claim 6, wherein medical images in the other apparatuses are inputted on an off-line basis.

10. The image information processing apparatus according to claim 6, wherein, when at least any one of an update, a change, an addition and elimination is carried out for the medical images and/or the supplemental information, only information changed for the registration is returned to its original position, and then, the medical images and/or the supplemental information is outputted to the other apparatus.

11. A medical image information processing system comprising the image information processing apparatus as described in claim 1.

12. The medical image information processing system described in claim 11, wherein the image information processing apparatus are installed in different hospitals.

13. The image information processing apparatus according to claim 2, wherein medical images in the other apparatuses are inputted on an on-line basis.

14. The image information processing apparatus according to claim 2, wherein medical images in the other apparatuses are inputted on an off-line basis.

15. The image information processing apparatus according to claim 2, wherein there is provided a means that returns only information changed for the registration to its original position when returning the medical images and/or the supplemental information to the other apparatus, in the case where at least any one of an update, a change, an addition and elimination is carried out, for the medical images and/or the supplemental information.

16. The image information processing method according to claim 7, wherein medical images in the other apparatuses are inputted on an on-line basis.

17. The image information processing method according to claim 7, wherein medical images in the other apparatuses are inputted on an off-line basis.

18. The image information processing apparatus according to claim 7, wherein, when at least any one of an update, a change, an addition and elimination is carried out for the medical images and/or the supplemental information, only information changed for the registration is returned to its original position, and then, the medical images and/or the supplemental information is outputted to the other apparatus.

19. The image information processing apparatus according to claim 8, wherein, when at least any one of an update, a change, an addition and elimination is carried out for the medical images and/or the supplemental information, only information changed for the registration is returned to its original position, and then, the medical images and/or the supplemental information is outputted to the other apparatus.

20. The image information processing apparatus according to claim 9, wherein, when at least any one of an update, a change, an addition and elimination is carried out for the medical images and/or the supplemental information, only information changed for the registration is returned to its original position, and then, the medical images and/or the supplemental information is outputted to the other apparatus.

21. The image information processing apparatus according to claim 16, wherein, when at least any one of an update, a change, an addition and elimination is carried out for the medical images and/or the supplemental information, only information changed for the registration is returned to its original position, and then, the medical images and/or the supplemental information is outputted to the other apparatus.

22. The image information processing apparatus according to claim 17, wherein, when at least any one of an update, a change, an addition and elimination is carried out for the medical images and/or the supplemental information, only information changed for the registration is returned to its original position, and then, the medical images and/or the supplemental information is outputted to the other apparatus.

23. A medical image information processing system comprising the image information processing apparatus as described in claim 2 and the other apparatuses are included.

24. The medical image information processing system described in claim 23, wherein the image information processing apparatus are installed in different hospitals.
